(19) United States
(12) Patent Application Publication
(10) Pub. No.: US 2020/0312461 A1
(43) Pub. Date: Oct. 1, 2020

(54) METHODS FOR SENSING AND ANALYZING IMPACTS AND PERFORMING AN ASSESSMENT

(71) Applicant: THE CLEVELAND CLINIC FOUNDATION, Cleveland, OH (US)

(72) Inventors: Adam Bartsch, Chaska, MN (US); Sergey Samorezov, Highland Hts., OH (US)

(21) Appl. No.: 16/720,589

(22) Filed: Dec. 19, 2019

Related U.S. Application Data
(60) Provisional application No. 62/781,986, filed on Dec. 19, 2018.

(51) Int. Cl. G16H 50/30; G06N 5/04; G06F 30/23; G16H 50/20

(52) U.S. Cl. CPC G16H 50/30; G16H 50/20; G06F 30/23; G06N 5/04

(57) ABSTRACT

Methods of analyzing and assessing users that have experienced impacts may include methods of identifying or filtering false positives, methods of co-registration of sensors, algorithms for translating sensed kinematics to relevant locations, and methods of assessing users based on historical and collected impact data combined with assessment data.

### CROSS-REFERENCE TO RELATED APPLICATIONS

The present application claims priority to U.S. Provisional Application No. 62/781,986 entitled Impact Sensing Techniques, and filed on Dec. 19, 2018, the content of which is hereby incorporated by reference herein in its entirety.

### TECHNOLOGICAL FIELD

The present disclosure relates to devices and systems for impact assessment. More particularly, the present disclosure relates to sensing and filtering impact data, analyzing the filtered impact data, and assessing the result of the impacts. Still more particularly, the present disclosure relates to adequately coupling sensors to a body part, co-registering the sensors, filtering out false positives, analyzing the sensed data, and assessing the sensed data to arrive at a clinically-based assessment.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Researchers and product developers have long since been trying to accurately and precisely sense impacts such as head impacts or other motion data occurring during sports, military activities, exercise, or other activities. While the ability to sense impacts has been available for some time, the ability to sense impacts with sufficient accuracy and precision to provide meaningful results has been more elusive. In the case of head impacts, the road blocks preventing such accuracy and precision include relative movement between the sensors and the head, false positive data, insufficient processing power and processing speed on a wearable device, and a host of other difficulties.

One solution to the relative movement issues has been to rely on a mouthguard that couples tightly with the upper teeth of a user and, as such, is relatively rigidly tied to the skull of a user. On the false positive front, mouthguards experience impacts in a lot of different contexts including users chewing on the mouthguard, dropping the mouthguards, throwing the mouthguards, etc. Normal use of a mouthguard may also include having it tethered to a helmet, which may cause the mouthguard to swing and contact the helmet or other objects. Mouthguards may also find themselves in gym bags, backpacks or other bags and may experience accelerations through handling of the bags.

Data processing power and processing speed continue to improve and be provided in smaller and smaller devices. As such, where a solution to false positives can be provided, further solutions for analyzing the accurate and precise data and assessing the meaning of the data are needed to meaningfully manage user activity.

### SUMMARY

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In one or more embodiments, a method of identifying false positive impact data using simulation may include sensing impact data including a linear acceleration and an angular acceleration, generating a simulation of motion of a body part of a user assumed to have been impacted to generate the impact data, and receiving footage of the user participating in the activity. The method may also include identifying the impact data as false positive data or true positive data based on a comparison of the simulation to the footage.

In one or more embodiments, a method of co-registration of a plurality of impact sensors configured for sensing the impact to a body part of a user may include performing an internal scan of a user and directly or indirectly measuring the relative position and orientation of the plurality of impact sensors relative to one another and relative to a selected anatomical feature based on the internal scan of the user.

In one or more embodiments, a method of assessing head impacts may include sensing impact data resulting from an impact to a user, generating a risk function from a set of historical and collected data including other impacts and clinical assessments and plotting the impact data against the risk function to arrive at an assessment of the user.

In one or more embodiments, a method of identifying true positive head impact data and filtering out other data may include sensing impact data and performing a first filtration operation based on a review of the impact data. The method may also include analyzing the impact data to determine resulting forces, kinematics at other locations, or other resulting factors to create analyzed data. The method may also include performing a second filtration operation based on a review of the analyzed data and identifying the impact data as preliminarily true positive data or false positive data.

In one or more embodiments, a method for modeling head impact data may include fitting an analytical harmonic function to the head impact data to generate an amplitude, a frequency, and a phase. The method may also include storing the type of analytical harmonic function and the amplitude, the frequency, and the phase.

In one or more embodiments, a method for calculation of six degree of freedom kinematics of a body reference point based on distributed measurements may include positioning a triaxial linear accelerometer and a triaxial angular rate sensor at a known point and sensing an impact with the accelerometer and rate sensor. The method may also include determining an acceleration at a location on or in the body away from the known point, wherein positioning comprises placing the rate sensor such that the sensitive axes of the rate sensor are aligned with the body anatomical sensitive axes.

In one or more embodiments, a method of determining an acceleration at a point of a body experiencing an impact may include sensing at least three linear accelerations with accelerometers arranged at a first point on the body and determining an acceleration at a second point on the body other that the first point. The determining may be performed by summing translational acceleration of the body with centripetal acceleration and tangential acceleration.

In one or more embodiments, a method for calculation of impact location and direction on a rigid, free body may include receiving linear and angular acceleration vectors of an impact at a reference point on the free body and establishing the direction of the impact as the direction of a linear acceleration vector. The method may also include establishing the location of the impact by calculating an arm vector originating at the center of gravity of the head and extending to a perpendicular intersection with a line of force and calculating an intersection of the line of force with a surface of the free body.

In one or more embodiments a method of assessing an impact on a body part may include sensing impact data from an impact on the body part and performing a finite element analysis on the body part based on the impact data. The method may also include identifying damage locations within the body part relating to the impact data and comparing the damage locations to clinical finding data to establish a model-based clinical finding.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a front view of model experiencing an impact on a model of a head, according to one or more embodiments.

FIG. 2 is a front view of a simulation of the motion experienced by the head due to the impact shown in FIG. 1, according to one or more embodiments.

FIG. 3 is a still frame of footage of a player experiencing a head impact.

FIG. 4A is a diagram of a method of identifying false positive impact data using simulation, according to one or more embodiments.

FIG. 4B is a diagram of a method of identifying false positive impact data using an analytical approach, according to one or more embodiments.

FIG. 4C is a diagram of a method of identifying false positive impact data using an analytical approach, according to one or more embodiments.

FIG. 5 is perspective view of a mouthpiece in place on a user and showing relative positions and orientations of the impact sensors relative to an anatomical feature or landmark of the user, according to one or more embodiments.

FIG. 6 is a diagram of a method of co-registering impact sensors, according to one or more embodiments.

FIG. 7 is a risk curve with a high range of uncertainty, according to one or more embodiments.

FIG. 8A is a risk curve with a lower range of uncertainty, according to one or more embodiments.

FIG. 8B shows a diagram of a method of assessing a user.

FIG. 8C shows a diagram of a method of assessing an impact on a body part.

FIG. 9A shows a diagram of linear acceleration vs. time of a non-head impact event.

FIG. 9B shows a diagram of angular velocity vs. time of a non-head impact event.

FIG. 9C shows a diagram of linear acceleration vs. time of another non-head impact event.

FIG. 9D shows a diagram of angular velocity vs. time of the another non-head impact event.

FIG. 10A shows a diagram of linear acceleration vs. time of a non-head impact event.

FIG. 10B shows a diagram of angular velocity vs. time of a non-head impact event.

FIG. 11A shows a diagram of linear acceleration vs. time of a non-head impact event.

FIG. 11B shows a diagram of angular velocity vs. time of a non-head impact event.

FIG. 12A shows a diagram of linear acceleration vs. time of an event that may be a head impact, but includes data that does not make sense for head motion.

FIG. 12B shows a diagram of angular velocity vs. time of an event that may be a head impact, but includes data that does not make sense for head motion.

FIG. 13A shows a diagram of linear acceleration vs. time for an event depicting a haversine shape.

FIG. 13B shows a diagram of a linear acceleration vs. time for an event where the amplitudes are nearing the 1-sigma imprecision of 400 rad/s².

FIG. 14A is a diagram of calculated accelerations at a center of gravity using a data transform algorithm.

FIG. 14B is a diagram of calculated accelerations at a center of gravity using an approach proposed by Zappa.

FIG. 14C is a diagram of a method of using a virtual sensor.

FIG. 14D is a diagram of a method of calculating a motion component at an arbitrary point.

FIG. 14E is a diagram of a method of calculating a impact direction and location.

FIG. 15 is a spatial diagram depicting variables associated with calculating kinematics at a point within a body.

FIG. 16 is a diagram depicting the variables associated with a linear accelerometer reading.

FIG. 17 is a diagram depicting the variables associated with calculating a direction and location of an impact force.

### DETAILED DESCRIPTION

The present disclosure, in one or more embodiments, relates several aspects of sensing impacts, analyzing the sensed data, and performing an assessment of the data. With respect to sensing impacts, co-registration of sensors may be performed prior to prepare the system to better analyze the data. Co-registration may be performed using particular measurement techniques such as magnetic resonance imaging (MRI), for example. With respect to analyzing the data, the present application discusses how to account for, reduce, or eliminate false positive results. That is, sensor data that is unlikely to be or clearly is not related to a head impact may be deemed irrelevant and discarded. In one or more embodiments, accounting for false positive sensor data may include a simulation approach, an analytical approach, or it may involve comparisons with other sensing devices. With further regard to analyzing the data, particular approaches to manipulating the sensed data to generate meaningful results based on a variety of factors such as repeated impacts, time between impacts, size of impact, and other factors may be used to arrive at meaningful results. Finally, with respect to assessment, the meaningful data and, in particular, meaningful data collected over time and combined with clinical or other assessment data, may be used to assess a user and provide a meaningful assessment based on a single impact. The assessment may include, for example, a risk curve, risk factor, or other metric by which a user may understand the severity and implications of a single impact while coaches, teams, trainers, or other managing persons or entities may make decisions based on the assessments.

Before getting into the details of the sensing, analyzing, and assessing, the present application is based on the availability of accurate and precise data. Such accurate and precise data may be provided by a mouthguard, for example, properly coupled to a user's upper jaw via the upper teeth. In one or more embodiments, a mouthguard may be provided that is manufactured according to the methods and systems described in U.S. patent application Ser. No. 16/682,656, entitled Impact Sensing Mouthguard, and filed on Nov. 13, 2019, the content of which is hereby incorporated by reference herein in its entirety.

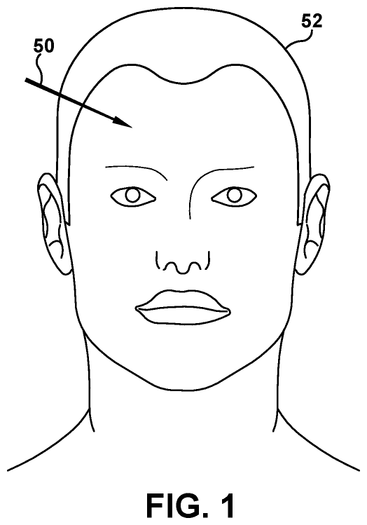

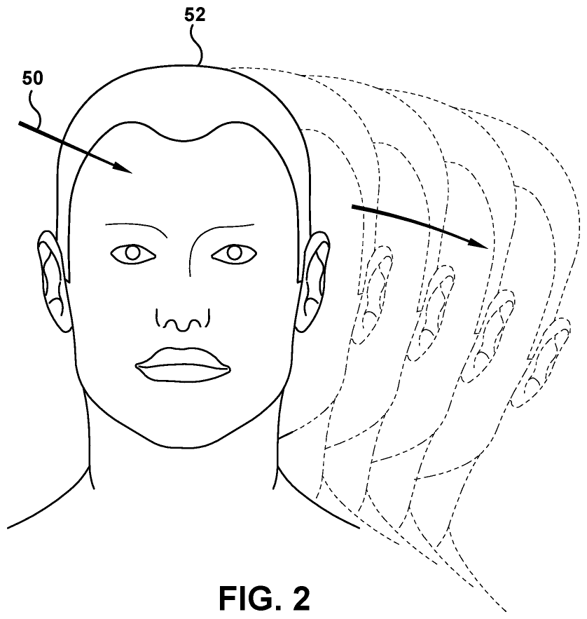

Turning now to FIGS. 1-4, an embodiment for identifying false positives is shown. As shown in FIG. 1, a force vector 50 is shown acting on a model of a head 52. The force vector may, for example, be a resulting force determined based on the sensed accelerations from a plurality of sensors. In FIG. 2, a simulation of the motion of the head is shown. That is, a simulation may be created based on a series of known factors in conjunction with the force vector and based on Newton's laws of motion. In one or more embodiments, the known factors may include the mass of the head, any restraints against motion such as the head connection to the neck, the strength of the neck, etc. As shown in FIG. 2, for example, the mathematical simulation of the head motion may suggest that the head translates to the left of the user and rearward as well as rotating counterclockwise and rearward relative to the user. While a force-based approach has been described, a kinematics approach that is based on recreating the sensed motion without consideration of forces acting on an object, may also be used.

In one or more embodiments, the animation motion based on the sensed data may be compared to actual visual and/or video evidence to help identify the sensed data as true positive data or false positive data. That is, as shown in FIG. 3, a still frame example of video footage of an impact is shown. As shown in FIG. 3, a ball carrier 54 in a football game has lowered his head to brace for impact of an oncoming defensive player 56. As shown, the helmets of the two players create an impact to both players. The impact is to the left/front side of the ball carrier's helmet and to the right/front side of the defensive player's helmet. If, for example, sensed data was received from a device on the defensive player 56 that resulted in a force vector as shown in FIG. 1, and a simulation shown in FIG. 2 at a same time that video footage of the defensive player 56 shows the impact of FIG. 3, it is likely that the sensed data is true positive data. That is, based on a review of the video footage shown in FIG. 3, it is likely that the defensive players head would shift to the left and rotate about his neck, which is consistent with the simulation of FIG. 2. Moreover, the actual video footage may be reviewed to determine if indeed the defensive player's head moved consistent with FIG. 2. When simulated motion is consistent with the witnessed impact, true positives may be much more likely and/or almost certain.

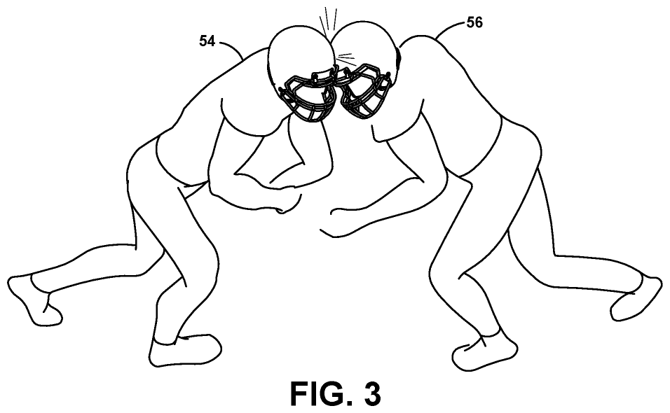

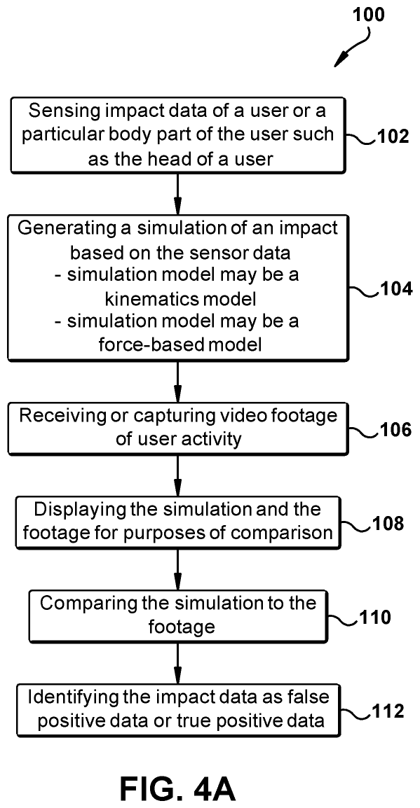

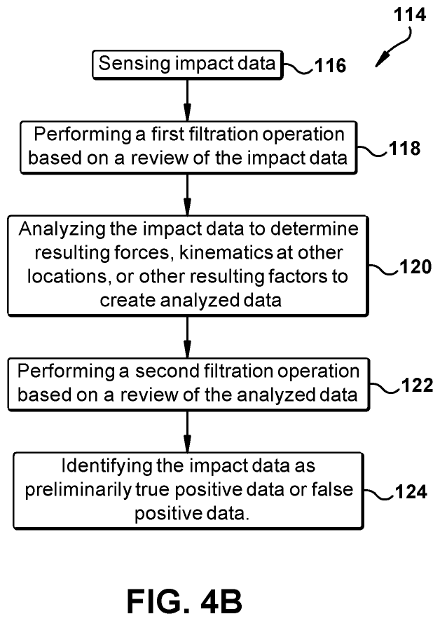

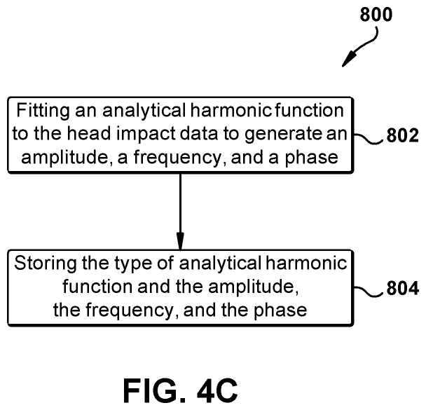

As shown in FIG. 4A, a method 100 of use may include sensing kinematics of a user or a particular body part of the user such as the head of a user. (102) The kinematics sensing may include sensing accelerations with one or more sensing devices such as accelerometers, gyroscopes, or other sensors. For example, sensing accelerations may include a sensing system capable of sensing motion in six directions or along six degrees of freedom (DOF) as a function of time during an impact. The sensors may sense linear accelerations along three orthogonal axes, such as X, Y, and Z. The sensors may also sense angular accelerations about each of the X, Y, and Z axes. Each sensor may be arranged along or about a selected axis and relative to the other sensors to create a six DOF sensing system.

The method may also include generating a simulation of an impact based on the sensor data. (104) That is, where the sensors are arranged on a mouthguard, for example, the sensor data may be assumed to be generated from an impact to the head of a user. Accordingly, a simulation of the head of a user may be generated based on the sensor data. In one or more embodiments, simulating an impact may be derived relatively directly from the sensor data. That is, a simulation model may be a kinematics model where the sensed accelerations over time are recreated and the effects of acceleration at one point on the head are used to calculate motion at other locations on the head. More particularly, the method may include computing/measuring the acceleration field of the skull, using equations of motion that connect the linear acceleration, angular acceleration, angular velocity and vector distances between measurement and calculation points on the head. In one or more embodiments, rigid body assumptions may be used such that relative positions of various points on the head remain in their relative positions throughout the motion.

In another embodiment, generating a simulation of an impact based on the sensor data may include a force-based approach where the sensor data is used in conjunction with measurements and/or assumptions of head mass, head geometry and mass moment of inertia to locate an impact force vector on the skull. In this embodiment, the impact force vector may be determined at or near the time of the peak linear acceleration. At or near the time of peak linear acceleration may be at a time plus or minus 5-10 milliseconds, for example.

The method may also include receiving or capturing video footage of user activity and, in particular, receiving or capturing video footage of impacts during user activity. (106) In one or more embodiments, a video system may be adapted to capture footage of a sporting event, for example, and monitor the footage for impacts such as by monitoring accelerations of motion involving either changes in direction or abrupt changes in speed. In one or more embodiments, the system may be adapted to create zoomed in replays of impacts on an automated basis for use in assessing impact data. In one or more embodiments, the system may be equipped with time stamp data that may be synchronized with or relatively closely tied to the sensing system so the time of impact data may be compared with video footage captured at a same or similar time. In one or more embodiments, the system may fetch footage based on a time stamp of the impact data and, for example, place a request to another system for footage at or near the time of the time stamp.

For purposes of comparison, the method may also include displaying the simulation and displaying the footage. (108) In one or more embodiments, the simulation and the video may be run consecutively (e.g., one after the other) or simultaneously (e.g., at the same time). The system may display the simulation and the footage side by side to allow for an efficient comparison. In one or more embodiments, the method may include prompting a user for an input with respect to the false positive or true positive nature of the impact data. That is, the method may include prompting the user to select between whether the sensed impact data appears to reflect a true positive impact or a false positive impact.

To determine whether impact data is false positive data or true positive data, a user or an automated system may perform a comparison. (110) For example, a user or an automated system may perceive a particular type of motion from the simulation. The user or an automated system may also review video footage of the activity at a same or similar time as the time the impact data was received. A comparison may be performed to determine whether the motion is sufficiently similar. In one or more embodiments, the comparison may simply involve determining whether there was an impact to the user at all. In this embodiment, a user or an automated system may review the footage to determine if there are any changes in direction or abrupt changes in speed. Alternatively or additionally, the comparison may involve comparing the type of motion by comparing the linear and rotational direction of motion. That is, the user or the automated system may review the footage to determine if the motion is in a particular direction or about a particular axis in a particular direction.

In one or more embodiments, the method may include identifying the impact data as false positive data or true positive data. (112) That is, where an automated system does the comparison, the system may identify the data as false positive data or true positive data. Where a human user does the comparison via the above-described display, for example, the system may store an input responsive to the prompt thereby identifying the impact data as false/true positive data.

While a simulation approach to false positive detection has been described, still other approaches may be used in addition to or as an alternative to the simulation approach. In one or more embodiments, devices may be used to assist in avoiding sensing of false positive impacts or to rule them out based without further analysis or study. For example, devices such as proximity sensors, light sensors, capacitive sensors may be used to eliminate sensed impacts when a mouthguard or other sensing device is not in the mouth or not on the teeth, for example. In one or more embodiments, these types of devices may include one or more of the devices described in U.S. patent application Ser. No. 16/682,656 entitled Impact Sensing Mouthguard, and filed on Nov. 13, 2019, the content of which is incorporated by reference herein in its entirety. Alternatively or additionally, multiple sensors or devices may be used to identify false positives. In one or more embodiments, multiple sensors may be used such as the systems described in U.S. patent application Ser. No. 16/682,787, entitled Multiple Sensor False Positive Protection, and filed on Nov. 13, 2019, the content of which is hereby incorporated by reference herein in its entirety. Alternatively or additionally, an analytical approach may be used where the data is analyzed to rule out false positives.

As shown in FIG. 4B, the analytical approach to ruling out false positives may include a method 114 of identifying true positives or ruling out false positives. In one or more embodiments, the method may include sensing impact data (116), performing a first filtration operation based on a review of the impact data (118), analyzing the impact data to determine resulting forces, kinematics at other locations, or other resulting factors to create analyzed data (120), performing a second filtration operation based on a review of the analyzed data (122), and identifying the impact data as preliminarily true positive data or false positive data (124). Each of these steps are discussed in more detail below.

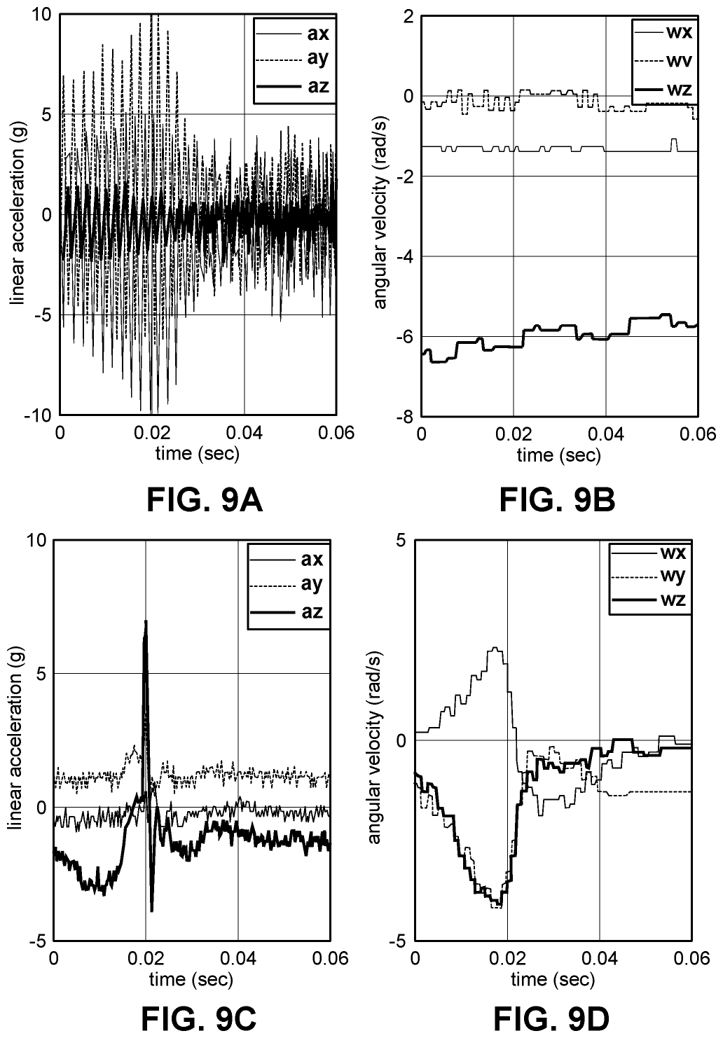

In one or more embodiments, the first filtration operation (118) may involve a review of the impact data to determine if it is an obvious non-head impact event. For example, where the impact data is a high amplitude short duration (e.g., 1 millisecond) spike with the rest of the signal near noise level, the data may be, for example, an acoustic signal, not a head impact as shown in FIGS. 9A and 9B. In another example, a high-frequency sign alternating acceleration time trace of approximately 60 milliseconds may also be quickly classified as a non-head impact event as shown in FIGS. 9C and 9D. This type of signal may be indicative of snapping a mouthguard onto a dentition, for example. Where the impact data is not deemed to be obvious non-head impact data, it may be preliminarily deemed true positive data and passed on for further analysis. Additionally or alternatively, the first filtration operation may involve comparing a time stamp of the impact data to a time stamp of an impact on a video. Here, if the time stamp of the impact aligns with an impact in the video, the impact data may be preliminarily identified as a true positive impact and passed on to further filters. Still other filtration procedures may be used with the raw impact data.

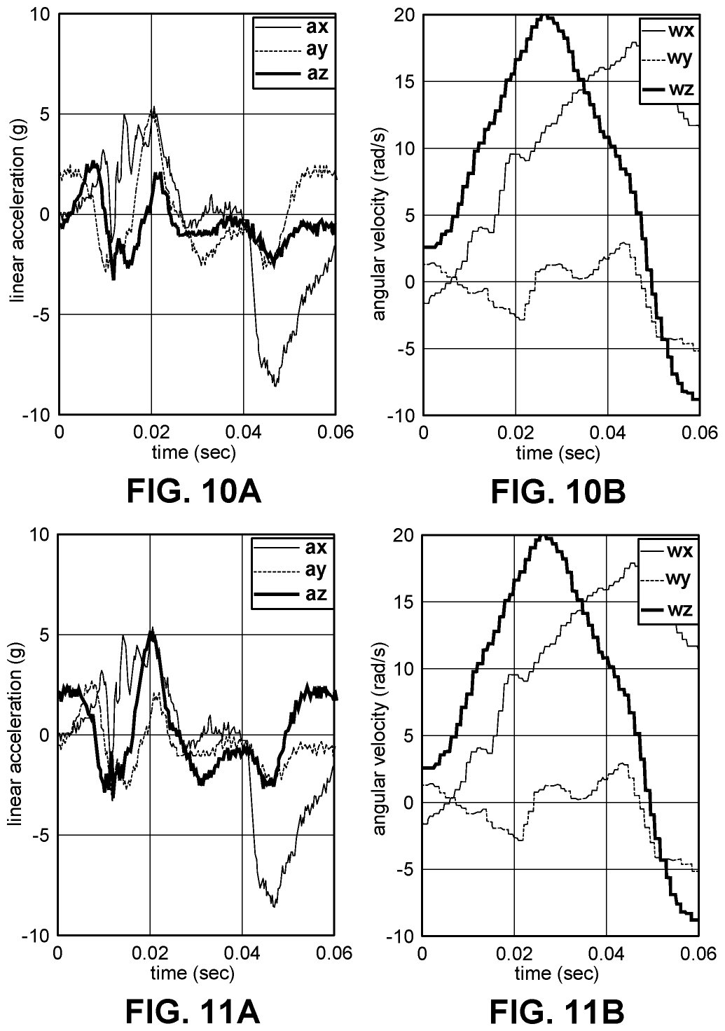

The second filtration operation (122) may involve several different approaches to performing filtration operations on analyzed data. In one or more embodiments, the impact data may be analyzed (e.g., at step 120) by transferring the data to the center of gravity of the head and the effects of the impact on the head may be analyzed (e.g., under step 122) to determine if data is likely or unlikely to be true positive impact data. In one or more embodiments, for example, the second filtration operation may include reviewing the transferred data to determine if it resembles a physically realistic head impact acceleration shape. If it does, the transferred data may preliminarily be deemed true positive data and be passed on to the next step. In one example as shown in FIGS. 10A and 10B, data from removal of a mouthguard is shown, which includes a kinematic signal that has amplitudes comparable to head impact. However, the shape of the linear acceleration pulses and timing of angular velocity pulses do not mimic physically realistic head motion. So, while this data may clear the first filtration operation the second filtration operation may identify the data as false positive data.

The system may also calculate an impact location and direction based on the impact data under step (120). In this embodiment, the second filtration operation (122) may include reviewing the calculated location and direction of impact and comparing it to a video of the impact believed to give rise to the impact data. If the location and direction of the impact are qualitatively similar to the video, the impact may be deemed preliminarily true positive data. One example of false positive data is shown in FIGS. 11A and 11B, which shows a boxer receiving impact to the left rear of the head directed toward the front when the video actually showed punches to both sides of the face. As such, despite similar time stamps, the impact data was deemed to be false positive.

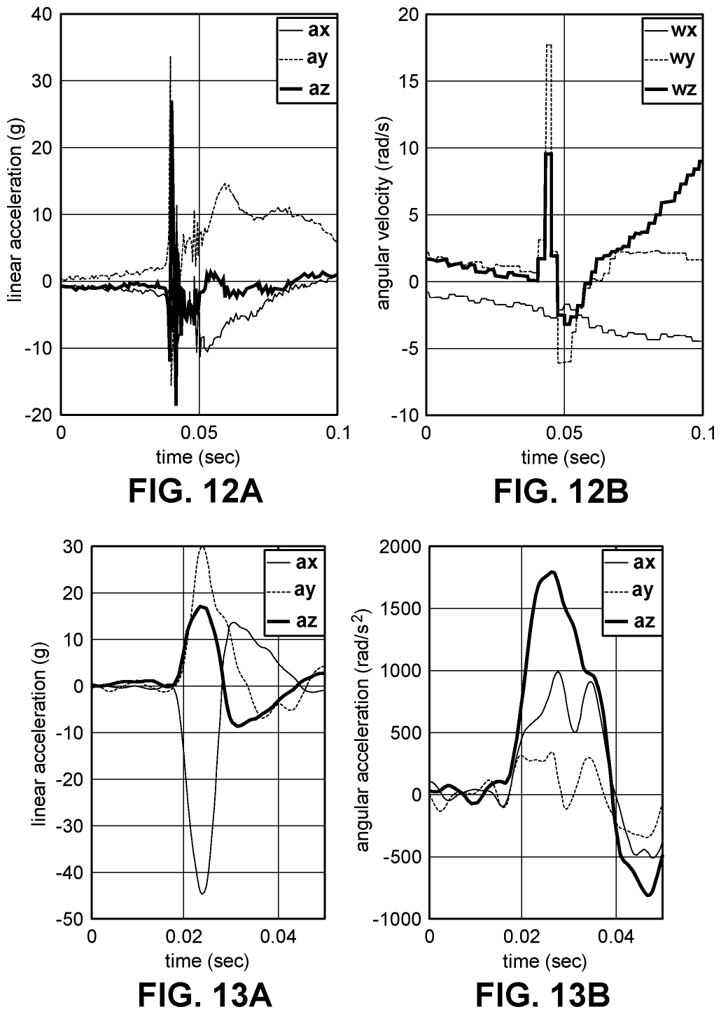

The system may also determine if motion calculated by the impact location, direction and kinematic traces (e.g., in the x, y, and z direction) of linear acceleration, angular acceleration, and angular velocity at the center of gravity of the head may obvious physical sense. If the calculated motion resembles known head impact motion, the impact may be deemed preliminarily true positive and be passed to the next filter. Where the an event pulse resembles physically realistic motion, but it is in tandem with information that does not make physical sense as shown in FIGS. 12A and 12B, the data may be determined to be false positive, otherwise, it may be deemed to be preliminarily true positive.

The system may also use ranges of spatial and temporal parameters to assist with the analysis. For example, the system may calculate spatial and temporal parameters and may compare the parameters to previously calibrated ranges. As shown in FIG. 13A, a haversine pulse-like shape in each axis is shown and a pulse time basis on the order 10 milliseconds is shown. In FIG. 13B, the amplitudes nearing the 1-sigma imprecision of 400 rad/s², the signal to noise ratio in angular acceleration decreases.

In one or more embodiments, the above analysis may be performed electronically, manually, or a combination of electronic and manual analysis may be provided. For example, in some embodiments, comparing the impulse wave shaped to a known true positive wave shape or range of wave shapes may be performed visually by a user. In other embodiments, an electronic system may compare the curves and may identify whether a curve falls within a range of curves or is close to a central curve or far from a central curve, for example. In one or more embodiments, an initial central curve or range of curves may be established and machine learning may be used to adjust the central curve or the range of curves over time based on continued input, sensing, and analysis. For example, an initial relatively small data set may be provided for establishing the central curve or range of curves that constitute true positive impacts. However, as additional information is collected, it may be determined that the initial set of data was somehow specific to the specimens or types of impacts use to establish the curves. As more and more data is input, the central curve or range of curves may be adjusted based on further knowledge of what constitutes a true positive. In one or more embodiments, true positive curves or ranges may be adjusted to accommodate different sports, age groups, athlete sizes, padded sports, helmeted sports, unpadded sports, bare knuckle sports, gloved sports, or other factors that are determined to affect the range of true positive curves.

In addition to the above-mentioned steps or procedures for ruling out false positives, the data may be more accurate when the sensors and/or systems of sensors are calibrated. Moreover, where false positives have been ruled out and the data is accurate, data compression may be a valuable tool for purposes of storage and transmission of data and may be well worth the effort knowing that the data that has been captured is strong meaningful data.

With respect to calibration, calibration of components can be done using shock towers in a drop test, pneumatic/hydraulic shaker table, etc. Calibration at a system level can be done with a crash dummy in a pneumatic impactor, monorail/twin wire drop tower or impact pendulum. Single degree of freedom tests (1DOF) or complex six degree of freedom tests (6DOF) can be used. In any calibration test a gold standard reference is used, and the calibration is applied algorithmically to the raw data received on the mouthguard. A calibration is successful when the post-calibrated outputs move towards higher accuracy and/or precision. In one or more embodiments, a method may include calibrating the individual sensors (gyro, accels). In another method the assembled circuit board can be calibrated. In another method the finished product can be calibrated. All calibration methods may involve a post-calibration input applied to the output data. This can be on a per-channel basis for raw voltage/digital outputs, or could be done as a final step in the computations for all data that has been processed. In one or more embodiments, calibration of the sensors may be performed to address differences relating to padded sports, unpadded sports, bare knuckle, elbow, or foot type sports and the like. In one or more embodiments, calibration may occur on the fly by comparing the ranges of impacts being sensed to known ranges for the various uses. For example, padded sports may include impacts with lower amplitudes and frequencies than unpadded sports and the system may calibrate on the fly after receiving a series of impacts that are more akin to a particular environment.

Regarding data compression, Frequency Content Algorithm may enable data compression and MEMS gyro Angular Acceleration Correction. Accurate concussion diagnosis may rely on accurate head impact kinematic data and sufficient amounts of kinematic data paired with clinically relevant behavioral deficits, blood tests, imaging or other quantitative medical data collection. Frequency Content Algorithm may be based on study of collisions, for which linear or angular velocity has an “S-shaped” time trace. One can approximate this curve through the harmonic content of its second derivative (first derivative=linear/angular acceleration, second derivative=linear/angular jerk). This approach allows for Harmonic based data compression, since the unique acceleration and velocity time traces can be represented simply by a few constants of an analytical equation instead of large files of digital sequences. This means impact signals that may require many thousands or tens of thousands of discrete points can be accurately approximated using three or six constants. This enables much larger volumes of impact data to be stored and reduces power/transmission requirements for wireless data transfer. The correction of linear/angular acceleration and linear/angular velocity over/under-prediction and for determination of empirical correction coefficients for sensors (accels, gyro) is often helpful due to the fact that miniature MEMS accelerometers and gyroscopes can remove signal amplitude due to OEM on-board filtering and limitations in sensor design. Inaccuracy in measured or computed linear and angular acceleration and velocity amplitude, frequency and phase may give a false impression of a head impact for both linear and rotational kinematics. Laboratory calibration methodology may include individual component calibration, algorithmic sensor output corrections, accurate determination of computational constants, system level linear pneumatic impactor tests, and the head form acceleration computations. In one or more embodiments, data compression may involve superimposing one, two, three, ten, twenty, or more linear time varying harmonics. Still other numbers of harmonics could be used. For example, constant values of multiple sine waves may be used to represent a curve. That is, an amplitude, frequency and phase for each sine wave may be stored together with a direction and location, for example. Still other approaches to data compression may be used.

While the fitting of harmonic functions to sensed impact data may be helpful for data compression, it may also be helpful for jumping between positional time traces, velocity time traces, acceleration time traces, and jerk time traces since all of these parameters may be related by derivatives or integrals. As such, the system may perform derivatives of harmonic time traces or integrals to arrive at corresponding time traces. Still further, fitting the harmonics to the data may allow for filtering, either as discussed with respect to false positives or for purposes of calibration for particular sports, for example. That is, where particular wave-shapes are known to be prevalent in some sports, but not others, filters may be used to capture wave-shapes that are relevant given a particular sport being participated in.

In one or more embodiments, as shown in FIG. 4C, a method 800 for modeling head impact data may include fitting an analytical harmonic function to the head impact data to generate an amplitude, a frequency, and a phase. (802) The method may also include storing the type of analytical harmonic function and the amplitude, the frequency, and the phase. (804) As may be appreciated, the several operations discussed above with respect to analysis using the harmonic function may be performed in conjunction with the above-mentioned method.

The more accurate and precise the impact data is in the above process, the more meaningful the simulation or any other analysis can be. One way to help improve the accuracy and precision of the impact data is to perform co-registration of the sensors. That is, while the sensors may be arranged on three orthogonal axes and may be adapted to sense accelerations along and/or about their respective axes, the sensors may not always be perfectly placed and obtaining data defining the relative position and orientation of the sensors relative to one another may be helpful. Moreover, while the sensors' positions relative to the center of gravity of a head or other anatomical landmark of the user may be generally known or assumed, a more precise dimensional relationship may allow for more precise analysis. Depending on the demands on the accuracy of the impact data, co-registration may be very advantageous. For example, calculated impact kinematics may vary 5-15% where co-registration is not performed. In one or more embodiments, where user anthropometry is relatively consistent across a group of users and assumptions about the anthropometry is used, the errors may be reduced to 5-10% where co-registration is performed based on the assumptions. For example, where a true impact results in a 50 g acceleration, the measured impact may be 45 g to 55 g. Where user-specific anthropometry, is used, the errors may be further reduced.

In one or more embodiments, co-registration may be performed by measuring. For example, measuring may include physically measuring the sensor position relative to user anatomy such as described in U.S. Pat. No. 9,585,619 entitled registration of head impact detection assembly, and filed on Feb. 17, 2012, the content of which is hereby incorporated by reference here in its entirety. In one or more embodiments, measuring may include directly measuring the positions and orientations using an internal scanning device. For example, in one or more embodiments, co-registration may be performed using magnetic resonance imaging (MRI) or computerized tomography (CT) where the user has a mouthpiece in place. Still other internal scanning devices may be used. In still other embodiments, measuring may include measuring the sensor locations relative to one another on a mouthguard and relating those positions to user anatomy using scans of user anatomy such as an MRI scan or a CT scan.

As mentioned, one embodiment may include a scan with a mouthpiece in place on a user. In the case of an MRI scan, due to the magnetic nature of the scan, metal objects may be avoided. In this case, a replica, model, or other mouthpiece closely resembling the construction of the mouthguard to be used by the user, may be used for the MRI scan. For example, a mouthpiece that is sized and shaped the same or similar to a mouthguard to be used may be created. Where the sensors are located in the mouthguard, the mouthpiece may include filler material in their place that is non-magnetic and, for example, shows up bright white, black, or some other identifiable color on an MRI. In one or more embodiments, a 3D printed replica circuit may be included in the mouthpiece. The 3D printed material may be water-like, for example, and may light up bright white on an MRI image in contrast to the surrounding tissue, teeth, and gums. In the case of a CT scan, the mouthguard with embedded functional circuitry and that the user plans to use may be used as the mouthpiece in the scan. Alternatively, a replica, model, or other mouthpiece may be used similar to the approach taken with the MRI.

In other embodiments, as mentioned, scans without the mouthpiece in place may be used. In one or more other embodiments, an MRI, CT, or other scan of a user may be performed without a mouthpiece in place and other techniques may be used to identify the location of the sensors relative to user anatomy. For example, a physical model (e.g., a dentition) of the user's teeth may be created. In this embodiment, measurements of the mouthguard may be used to identify sensor locations/orientations relative to one another. Scans of the mouthguard on the dentition such as MRI scans, CT scans, 3D laser scans or other physical scans may be used to identify the relative position and orientation of the sensors to the dentition or markers on the dentition. The MRI or CT scan of the user may then be used to identify the relative position of the sensors to the user anatomy using markers on the head and the dentition. In one or more embodiments, bite wax impressions may be used to get impressions of the teeth. Additionally or alternatively, the impressions may be classified into maxillary arch classes such as class I, II, or III.

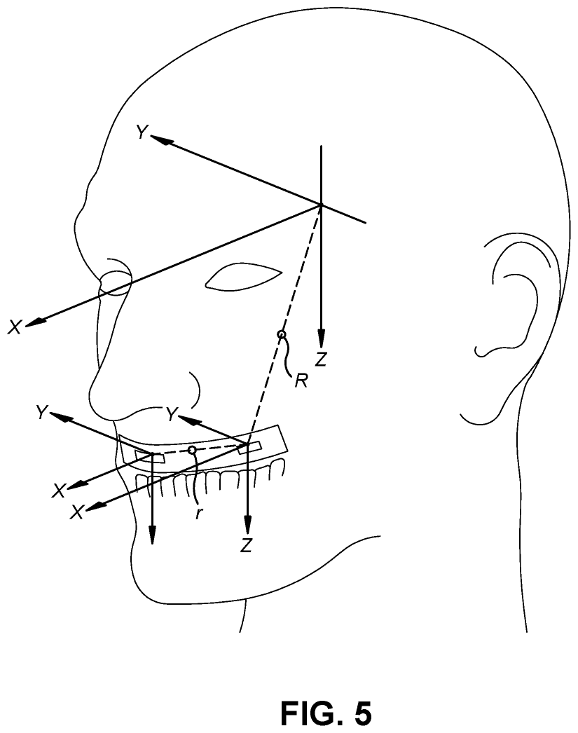

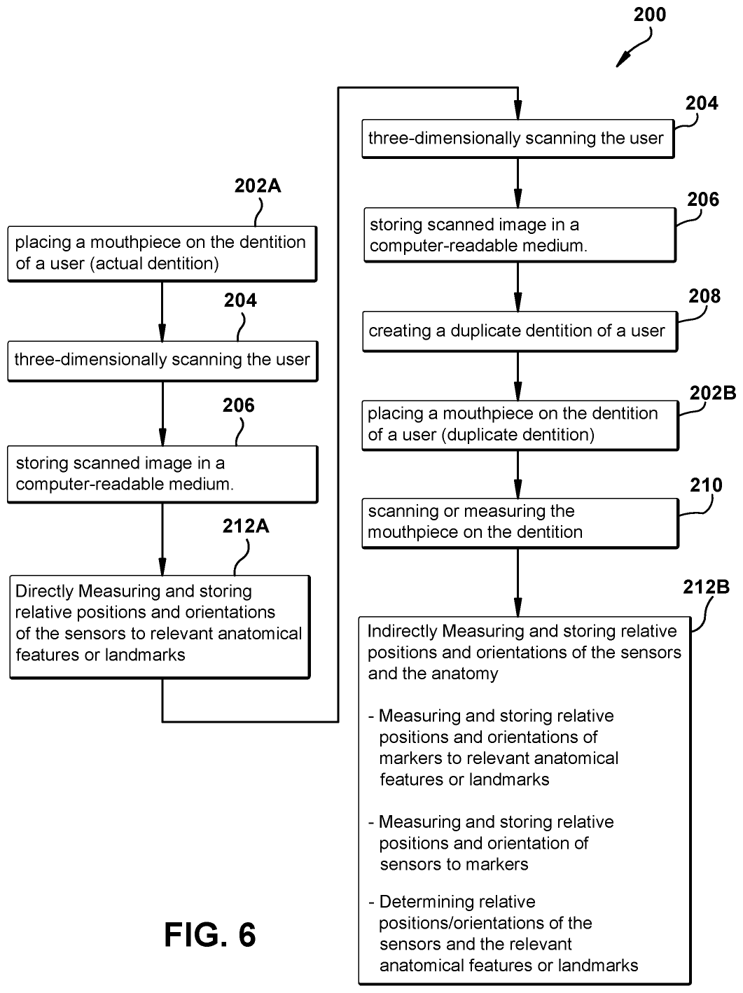

In one or more embodiments, and with reference to FIG. 6, a method 200 of co-registration may be provided. The method 200 may include placing a mouthpiece on a dentition of a user (202A/202B). In one or more embodiments, this step may include placing the mouthpiece in the user's mouth (202A). Alternatively or additionally, placing the mouthpiece on a dentition of the user may include placing the mouthpiece on a duplicate dentition of the mouth of a user (202B). The method may also include three-dimensionally performing an internal scan of the user (204). This step may be performed with the mouthpiece in place in the user's mouth or without the mouthpiece in the mouth of the user. In either case, the scanned image may be stored in a computer-readable medium (206).

Where the mouthpiece is in the mouth during scanning, the relative positions and orientations of sensors and anatomy may be measured and stored directly (212A). For example, and as shown in FIG. 5, the relative positions (r) and orientations of the sensors may be ascertained from the image to verify, adjust, or refine the relative positions and orientations of the sensors relative to one another. It is to be appreciated that where the actual mouthguard is being used during the scan, manufacturing tolerances associated with sensor placement may be accounted for during co-registration by measuring the actual position and orientation of the sensors. Moreover, and with respect to direct measurement of sensor positions, the images may be used to measure the positions and orientations of the sensors relative to particular anatomical features or landmarks. For example, in one or more embodiments, the relative position (R) of the sensors and the relative orientation of the sensors with respect to the center of gravity of the head or with respect to particular portions of the brain may be measured and stored.

Where the mouthpiece is not in the mouth during scanning, the relative positions and orientations of sensors and anatomy may be measured and stored indirectly (212B). That is, the relative positions of markers on the anatomy may be stored based on the scan of the user. For example, marker locations on the user's teeth relative to particular anatomical features or landmarks such as the center of gravity of the head may be stored. Further, where the mouthpiece is not placed in the mouth during the scan of the user, the method may include creating a duplicate dentition of the user's mouth. (208) This may be created from the MRI/CT scan using a 3-dimensional printer, using bite wax impressions, or using other known mouth molding techniques. The mouthpiece may be placed on the duplicate dentition and physical measurements of the sensors relative to markers on the dentition may be taken. (210) Additionally or alternatively, scans such as laser scans, MRI scans, CT scans or other scans of the mouthpiece on the duplicate dentition may be used to identify the sensor locations relative to the markers on the dentition. (210) The markers on the duplicate dentition may coincide with the markers used in the MRI/CT scan of the user. As such, the method may include indirectly determining the positions and orientations of the sensors relative to the anatomical features or landmarks of interest, such as the center of gravity of the head, by relying on the markers tying the two sets of data together. (212B)

The impact data may be analyzed to determine kinematics, forces, or other values at or near the sensed location, at particular points of interest in the head (e.g., head center of gravity), or at other locations. In one or more embodiments, rigid body equations or deformable body equations may be used such as those outlined in U.S. Pat. Nos. 9,289,176, 9,044,198, 9,149,227, and 9,585,619, the content of each of which is hereby incorporated by reference herein in its entirety.

In one or more embodiments, the methods of transferring the location of sensed accelerations from one location to another may be based on methods used by Padgaonkar and Zappa. In one or more embodiments, particular approaches may include taring raw data to remove initial sensor offsets. This may help ensure that each impact is computed as the overall change in head motion. Other methods could use the initial conditions, for example, being able to compute an initial velocity/orientation before the head begins substantial acceleration after impact. In one or more embodiments, the algorithms may be sport-specific algorithms and false positive settings can be employed where a user can change on the fly (e.g. helmeted vs. non-helmeted impacts).

The methods described herein may be used with a variety of different sensor systems and arrangements. For example, a system for measuring 3 linear accelerations and 3 angular rates may be provided. Still further systems for measuring six, nine, or twelve linear accelerations with 3 angular rates, may be provided. In one or more embodiments, the system may differentiate a gyroscope signal to get an angular acceleration. Still further, knowledge of filtering based on representations of kinematics signals in terms of jerk, acceleration, and velocity may be provided where a second accelerometer may help with iterations. A system of 12 linear accelerometers may also be used and methods based on Padgoankar, Zappa, and/or a virtual sensor measurement scheme may be used. In one or more embodiments, the system may auto-reconfigure the algorithm, perform calibration, and perform co-registration when a user changes sports.

Human data that is acquired for purposes of clinician examination is preferably of high accuracy and precision or it may lead to clinical uncertainty. A head impact monitor measures head kinematics during collision in athletic events, using sensors embedded in an athlete's mouthguard. For sensors to fit in the mouthguard, the sensors may be distributed along the dentition (instead of being lumped in one spot), and there is no textbook head kinematics solution for this arrangement.

The Data Translation Algorithm may include a computation of a “virtual sensor measurement” at any selected reference point and then may compute head kinematics using a more common solution. The Data Translation Algorithm enables impact monitor Mouthguard sensors to be specifically distributed along the athlete's dentition within the confines of a mouthguard and reduces or eliminates directional sensitivity in measurements. By reducing or eliminating directional sensitivity, and by having freedom to place sensors nearly anywhere inside the mouthguard, measurement accuracy and precision may be enhanced and hardware design remains flexible. This method may be particularly advantageous for the mathematically sufficient “12a” approach, where ideas from Zappa and Padoangkar are used with four linear accelerometers in a non-coplanar arrangement. In one example, 12a instrumented mouthguard outputs were the result of direct measurement by an accelerometer array and follow-on custom computational data translation algorithm (DTA), which relied on accurate knowledge of design-related computational constants. Zappa et al. (2001) shows that 12a non-coplanar accelerometer configuration theoretically allows for algebraic computations of head linear and rotational kinematics, as time-varying vectors, based on the equation for acceleration of a point on a moving rigid body. The rigid body relationship is described in equation below, where rₚ is a vector of constant length between a point O and point P, a₀ is linear acceleration of a point O on the body, angular velocity is ω, and angular acceleration is \dot{ω}.

σₚ=a₀+\dot{ω}×rₚ+ω×(ω×rₚ)

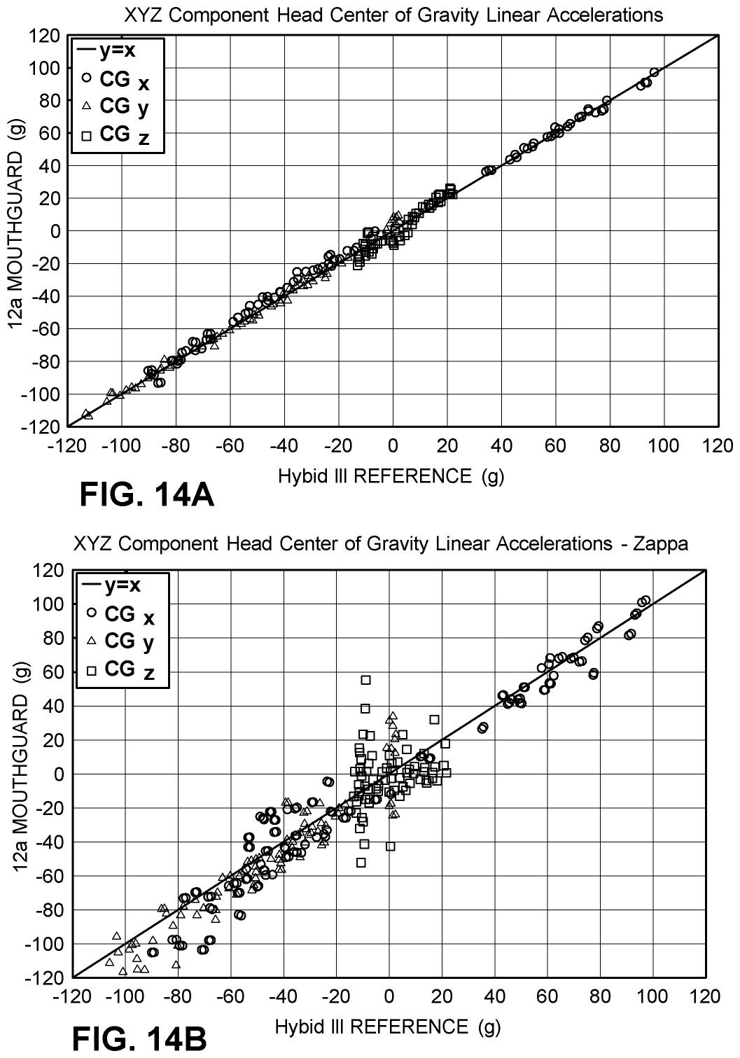

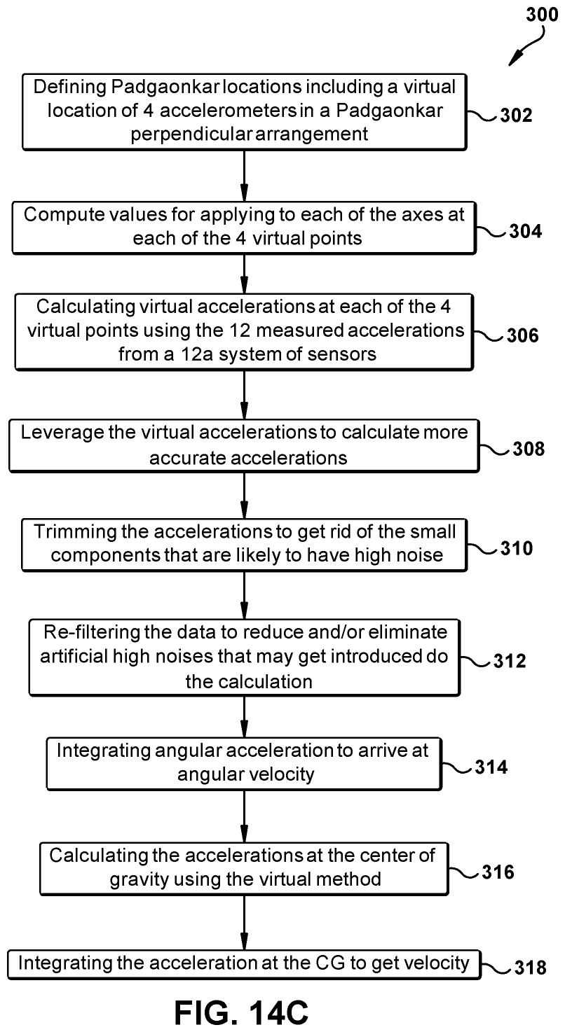

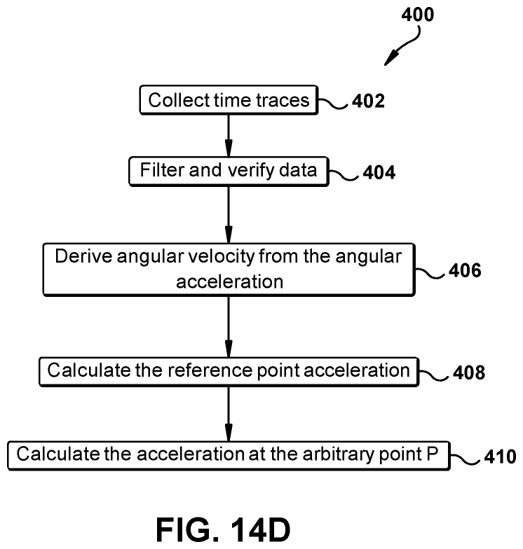

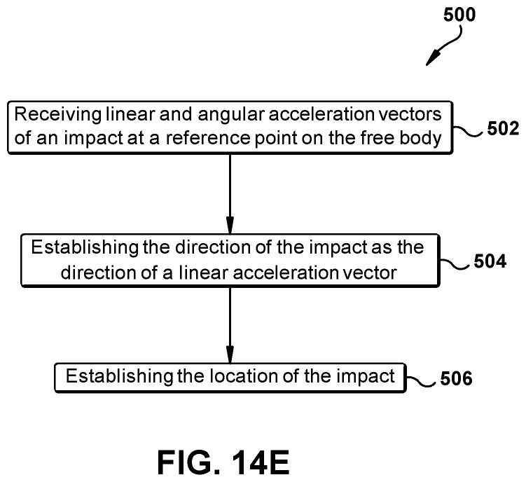

In the DTA computations there are implicit assumptions of a skull moving as a rigid body and of adequate coupling of instrument to the skull. The computational method may not be valid for situations when the skull is significantly deformed or instrument to skull coupling is inadequate. The DTA is focused on accurate estimates of time-varying vectors of head acceleration at the beginning stages of impact, where accelerations are high but velocities are still low. The improvement in “virtual acceleration” DTA v. Zappa is self-evident when reviewing FIGS. 14A and 14B.

The general solution for translating sensed data from a sensor location to another point in the head such as the center of gravity of the head is described below. However, when combined with the virtual sensor technique, still further accurate results may be achieved.

In one or more embodiments, for example, and as shown in FIG. 14C, a method for translating impact data to another point may employ a method 300 using a virtual sensor. The method may include defining Padgaonkar locations including a virtual location of 4 accelerometers in a Padgaonkar perpendicular arrangement. (302) These points may be with respect to the head center of gravity at point (0,0,0). In one or more embodiments, the points may include:

P_{REF}(60,0,60),

P_{X}(70,0,60),

P_{Y}(60,10,60), and

P_{Z}(60,0,70).

Using a Padgaonkar method, values are computed for applying to each of the axes at each of the 4 virtual points. (304) In one or more embodiments, the values may include:

v_{pREF}=[−0.1384,0.5056,0.2202,0.4125];

v_{pX}=[0.0916,0.6720,−0.0980,0.3345];

v_{pY}=[−0.1156,0.4960,0.0581, and 0.5614];

v_{pZ}=[0.9076,−0.4611,0.1566, and 0.3969].

The method may also include calculating virtual accelerations at each of the 4 virtual points using the 12 measured accelerations from a 12a system of sensors (e.g., a1, a2, a3, and a4, each having 3 axes.) (306) In code form the accelerations may be calculated as follows:

aPREF=zeros(size(a2));

aPX=zeros(size(a2));

aPY=zeros(size(a2));

aPZ=zeros(size(a2));

for n=1:length(t)

aPREF(:,n)=[a2(:,n)a1(:,n)a4(:,n)

a3(:,n)]*vpREF;

aPX(:,n)=[a2(:,n)a1(:,n)a4(:,n)

a3(:,n)]*vpX;

aPY(:,n)=[a2(:,n)a1(:,n)a4(:,n)

a3(:,n)]*vpY;

aPZ(:,n)=[a2(:,n)a1(:,n)a4(:,n)

a3(:,n)]*vpZ;

The method may leverage the virtual accelerations to calculate more accurate accelerations. (308) In one or more embodiments, the method may include trimming the accelerations to get rid of the small components that are likely to have high noise. (310) This may be performed for both real/measured components and virtual components of linear/angular acceleration/velocity. In code form, this may calculated as follows:

omdP=zeros(size(a2));

omdP(1,:)=(aPY(3,:)−aPREF(3,:))/(PY(2)−PREF(2));

omdP(2,:)=−(aPX(3,:)−aPREF(3,:))/(PX(1)−PREF(1));

omdP(3,:)=(aPX(2,:)−aPREF(2,:))/2/(PX(1)−PREF(1))−

(aPY(1,:)−aPREF(1,:))/2/(PY(2)−PREF(2));

The method may also include re-filtering the data to reduce and/or eliminate artificial high noises that may get introduced do the calculation. (312) In one or more embodiments, the method may include post-computation filtering on (1) differentiation of gyroscope angular rate to get angular acceleration, (2) post-virtual measure calculation, (3) post-CG calculation, and so on. In one or more embodiments, anywhere that the a calculation is performed, the method may include re-filtering the data in a manner similar to the manner used to filter the input data. In one example, the gyroscope angular rate data may be filtered at 200 Hz. The data may be differentiated to arrive at an angular acceleration and that result may be re-filtered at 200 Hz, and then the angular acceleration at the CG may be computed and re-filtered at 200 Hz. One example of re-filtering is shown here:

omdP(1,:)=filtfilt(B,A,omdP(1,:));

omdP(2,:)=filtfilt(B,A,omdP(2,:));

omdP(3,:)=filtfilt(B,A,omdP(3,:));

omdPMag=sqrt(omdP(1,:)·\hat{ }2+omdP(2,:)·\hat{ }2+omdP(3,:)·\hat{ }2);

In one or more embodiments, the method may include integrating angular acceleration to arrive at angular velocity. (314) That is, where a 12 a approach is used, angular acceleration may be calculated using multiple linear accelerations and integration of the angular acceleration may be used to determine angular velocity (e.g., rather than measuring it with a gyroscope).

The integration may be performed as follows:

omP=zeros(size(omdP));
for ii=1:3

omP(ii,:)=cumtrapz(omdP(ii,:))*dt;

end

omPMag=sqrt(omP(1,:).\hat{ }2+omP(2,:).\hat{ }2+omP(3,:).\hat{ }2);%resul
tant

omPchange=max(omPMag)−min(omPMag);

The method may also include calculating the accelerations at the center of gravity using the virtual method. (316) The inputs may include angular accelerations and an angular accelerations that are more accurate by virtue of the virtual method.

acgP2=zeros(size(a2));

acgP2tang=zeros(size(a2)); acgP2centr=zeros(size(a2));

for n=1:length(t)

acgP2tang(:,n)=cross(omdP(:,n),(−A2));

acgP2centr(:,n)=cross(omP(:,n),cross(omP(:,n),(−A2)));
acgP2(:,n)=a2(:,n)+acgP2tang(:,n)+acgP2centr(:,n);

end

acgP2mag=sqrt(acgP2(1,:).\hat{ }2+acgP2(2,:).\hat{ }2+acgP2(3,:).\hat{ }2
);

Acg=acgP2;
AcgMag=acgP2mag;

In addition, the method may include integrating the acceleration at the CG to get velocity at the CG as follows (318):

vcgP2=zeros(size(acgP2));
for ii=1:3

vcgP2(ii,:)=cumtrapz(acgP2(ii,:))*dt;

end

vcgP2mag=sqrt(vcgP2(1,:).\hat{ }2+vcgP2(2,:).\hat{ }2+vcgP2(3,:).\hat{ }2
);

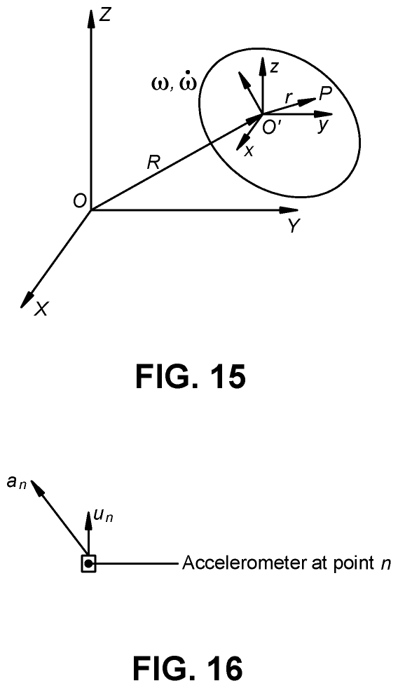

A more detailed discussion of the algorithm behind translating the sensed data to the center of gravity of the head or to another relevant location may be had with respect to FIG. 15. FIG. 15 depicts a free body moving in a global coordinate system OXYZ. Vector R indicates a body reference point O′ position relative to point O. Vectors ω and \dot{ω} indicate body angular velocity and angular acceleration, respectively. There is also a body fixed coordinate system O′xyz and an arbitrary point P on the body. The body is presumed to be rigid such that the point P position in O′xyz coordinates does not change. The resulting body movement in OZYX coordinates is presumed to be a sum of translation of point O′ and rotation around point O′. In addition, as shown, OO′=R (e.g., position of the moving body in the global coordinates). The variable w is the angular velocity of the body. The variable O′P=r (e.g., position of arbitrary point P on the body in the body fixed coordinate system). Finally, OP=rP (e.g., position of point P in global coordinates)

For a point P on a body in OXYZ coordinates:

Position rₚ=R+r (1)

Velocity νₚ=\dot{R}+\dot{r}=R+(\dot{r})ᵣ+ω×r (2)

where x denotes vector cross product, \dot{r} denotes time derivative of r

Acceleration a_{p=\ddot{R}}+(\ddot{r})ᵣ+2ω×(\dot{r})ᵣ+ω×(ω×r)+\dot{ω}×r (3)

Eliminating deformable body terms aₚ=\ddot{R}+ω×(ω×r)+\dot{ω}×r (4)

As shown in Equation (4), acceleration of point P is a sum of translational acceleration \ddot{R} and two components related to rotation—centripetal acceleration ω×(ω×r), and tangential acceleration \dot{ω}×r.

For a mouthguard-based measurement scheme, not all variables on the right side of equation (4) may be known. Vector ω may be measured directly by an angular rate sensor, also known as a gyroscope. Vector r is known and is constant in O′xyz for a given point P. Vector \dot{ω} is a time derivative of ω and may be derived from ω. While not necessarily apparent, a mouthguard may include a sensor configuration that does provide for direct measurement of \ddot{R} (translational acceleration of point O′) and a detailed discussion of this is provided below.

With respect to angular velocity, ω, and the direct measurement thereof, angular velocity is a free vector. Angular velocity of the body at point O′ is equivalent to that measured at point P, or any other point on the body. Therefore, knowledge of angular rate sensor position is not as important as knowledge of its orientation.

If the sensitive axes of the angular rate sensor are known to be collinear with axes defined by the intersection of the anatomical mid-sagittal and Frankfurt planes (atypical case) then no static angular correction is needed. But for the general case, the angular rate sensor sensitive axes may be assumed to be mis-aligned with the anatomical axes. To express the angular velocity vector in the desired anatomical axes using the output of the angular rate sensor, one needs to know the angular rate sensitive axes' orientations with respect to the anatomical axes and perform a static angular correction.

The simplest way to obtain vector \dot{ω} is through numerical differentiation of measured ω. Another approach is through positioning of an array of 12 accelerometer axes at 4 non-coplanar points (which can be simplified through clever positioning into 9 orthogonal accelerometer axes sensing at 4 points). This method has an implicit requirement of sufficiently high measurement bandwidth, sampling rate and low noise in ω. When this is not the case, an analytical fit of measured ω may be considered, with \dot{ω} obtained through analytical differentiation. This method may rely on a priori knowledge of the type of impact in the spatial and temporal domains such as through in vitro laboratory testing with instrumented surrogates or via empirical determination of characteristic impacts for a given sport. For example, if it is known that the sensor is anticipated as being exposed to helmeted impacts or if it is know that the sensor is anticipated as being exposed to non-helmeted impacts, the computation method can be adjusted to properly treat the data. For example, for helmeted impacts, the method may filter angular velocity and angular acceleration at approximately 200 Hz. For barehead impacts, the filter may be closer to 400 Hz, for example.

With knowledge of r, ω, and \dot{ω}, the value \ddot{R} may be determined if the acceleration at a point is known. In order to understand the relationship of \ddot{R} to the measured accelerations and angular velocities the acceleration at points P may be measured. As an initial note, and with reference to FIG. 16, the output of an accelerometer measurement in acceleration units, typically given in acceleration scaled by the force of gravity or ‘g’, is a time series of scalar values, which are determined by:
1. true acceleration vector (an) at the time and place (point ‘n’) of measurement; and
2. orientation of accelerometer sensitive axis (un) relative to the acceleration vector

If an accelerometer is placed at a point n, such that accelerometer sensitive axis orientation is given by unit vector uₙ, and true acceleration of the point n is aₙ, then the accelerometer output aₙₘ in acceleration units is a dot product

aₙₘ=aₙ·uₙ (5)

Where a mouthguard prototype has three accelerometer sensitive axes, that may or may not be orthogonal to each other, with known positions r₁, r₂, r₃, and unit vectors of their sensitive axes u₁, u₂, and u₃, then true acceleration at point n is given by equation (4) when P=n. Applying equation (5) results in the following:

aₙₘ=aₙ·uₙ=(\ddot{R}+ω×(ω×rₙ)+\dot{ω}×rₙ)·uₙ

aₙₘ=\ddot{R}·uₙ+(ω×(ω×rₙ))·uₙ+(\dot{ω}×rₙ)·uₙ (6)

Therefore, the measured output of an accelerometer includes components related to both translational and rotational acceleration. These components are separable.

Vector R can be determined using equation (6) as follows. Taking all known quantities, as a result of mouthguard measurement at a given moment in time, in the equation (6) right side, obtain

\ddot{R}·uₙ=aₙₘ−(ω×(ω×rₙ))·uₙ−(\dot{ω}×rₙ)·uₙ (7)

Also, for a given moment in time for a given accelerometer sensitive axis n=1,2,3

\dot{R}·uₙ=Constantₙ(t) (8)

This equation is linear in \ddot{R}_{X}, \ddot{R}_{Y}, \ddot{R}_{Z}, which are X, Y, Z components of the vector \ddot{R}

\ddot{R}=\ddot{R}_{X}i+\ddot{R}_{Y}j+\ddot{R}_{Z}k,

where i, j, k are unit vectors of the coordinate system.

Expressing accelerometer sensitive axis unit vectors in the OXYZ coordinate system as

uₙ=u_{nX}·i+u_{nY}·j+u_{nZ}·k, equation (8) can be rewritten as

\ddot{R}_{X}·u_{nX}+\ddot{R}_{Y}·u_{nY}+\ddot{R}Z·u_{nZ}=Constantₙ(t) n=1,2,3 (9)

Data from each of the three mouthguard accelerometer sensitive axes and the angular rate sensor can be used to generate three linear equations of the form (9) for accelerometer sensitive axis positions n=1, 2, 3. If these equations are linearly independent, then the three coordinates of vector \ddot{R} for any given moment in time can be calculated. The condition of linear independence is equal to the requirement that determinant of matrix (10) is non-zero.

$$( \begin{matrix}{u}_{1 X} & {u}_{1 Y} & {u}_{1 Z} \\ {u}_{2 X} & {u}_{2 Y} & {u}_{2 Z} \\ {u}_{3 X} & {u}_{3 Y} & {u}_{3 Z}\end{matrix} ) \qquad ( 10 )$$

This means that any two mouthguard accelerometers may preferably not have parallel sensitive axes. From a practical standpoint, computational errors in value of \ddot{R} would be minimized if this determinant has maximum value. Considering that the length of a unit vector is 1, this condition would be satisfied if matrix (10) is a unity matrix

$$( \begin{matrix}1 & 0 & 0 \\ 0 & 1 & 0 \\ 0 & 0 & 1\end{matrix} ) \qquad ( 11 )$$

and all three mouthguard accelerometer axes have mutually orthogonal sensitive axes.

If all three accelerometer sensitive axes can be placed at point O′, then components of the vector \ddot{R} can be measured directly and with possible need for angular correction in the same manner described previously for the angular rate sensor.

In light of the above, equation (4) can be used to determine the acceleration of an arbitrary point on a free moving body.

a_{P}=\ddot{R}+ω×(ω×r)+\dot{ω}×r (4)

where:

r is the point position vector on the body (constant during collision in a body Reference frame),

ω is measured vector of angular velocity

\dot{ω} is angular acceleration, derived from measured ω

\ddot{R} is calculated translational acceleration of Reference point O′; it is a solution of a system of 3 linear equations with 3 unknowns (9) for each moment in time. The coefficients in these equations are based on measured values of linear acceleration in three locations, measured angular velocity, derived angular acceleration \dot{ω}, and known positions and orientations of the mouthguard sensitive axes.

In operation and use, the system may include stored or input values of locations and orientations of sensors. As shown in FIG. 14D, and in a method 400 of calculating a motion factor at an arbitrary point, the system may collect time traces (402) and the data may be filtered and data verified (404). From the angular velocity, the angular acceleration may be derived (406) and the reference point acceleration may be calculated (408). Using equation (4), the acceleration at the arbitrary point P may be calculated (410). It is to be appreciated that the approach may be used with a wide variety of sensor arrangements. In particular, the approach may be used with a 3 linear accelerometer and 3 angular rate sensors, but may also be used with 12 linear accelerometers, for example. Moreover, the method may be used without or in conjunction with the virtual sensor method 300.

In one or more embodiments, the system may consider corrections based on the sensors position and orientation during an impact. However, in other embodiments, the errors associated with this change may be deemed tolerable.

While accelerations at one or more points of interest may be valuable in assessing head impacts, the impact direction and location may also be valuable. It may be common when analyzing head impacts to assume that impact vectors from impacts pass through the center of gravity of the head. However, in many cases, they do not.

In one or more embodiments, an assumption may be made that head movement is similar to a free rigid body in an initial stage of collision and, as such, effects of a connected neck or other restraints may be ignored at least with respect to the initial state of collision when acceleration is rising to its peak value, for example. In one or more embodiments, experience-based guesses about mass moment of inertia and skull geometry may be used to arrive at a recursive algorithm to estimate the location of a collision force on the skull. This may accurately predict impact direction and location on the skull. For example, an uppercut will display impact to the chin in the upward direction, while prior systems may show such a blow as passing through the neck and the center of gravity of the head.

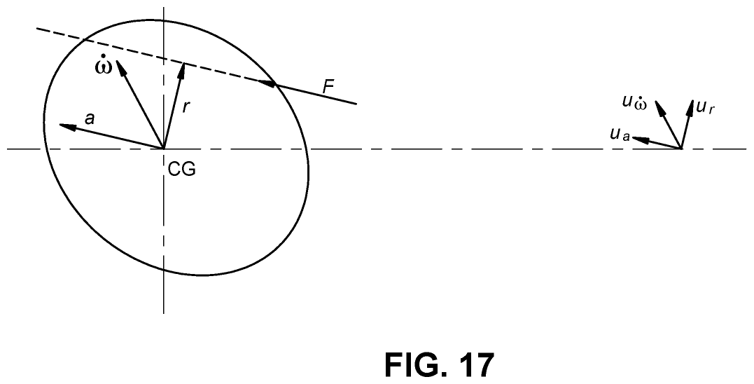

Referring to FIG. 17, a force F applied at an arbitrary point on a surface of a free body of mass m and of mass moment of inertia Iₘ, may cause linear acceleration at the body center of gravity a_{cg} and angular acceleration \dot{ω}. Vector r originates at CG and is perpendicular to the line of force F action.

Each of these vectors can be represented generally as a product of a unit vector uᵢ, which determines the vector direction, and scalar magnitude mod(i), which determines the vector length,

F=u_{F}*mod(F);

r=uᵣ*mod(r);

a_{cg}=uₐ*mod(a_{cg});

a=uₐ*mod(\dot{ω});

By definition

u_{F}=uₐ; (1)

uᵣ×uₐ=u_{wdot} (2) x denotes vector cross product

At the same time,

F=m*a_{cg} (3)

and

r×F=Iₘ*\dot{ω} (4)

Presuming that for a given moment in time, for example the time of peak value, both magnitudes and unit vectors for linear and angular acceleration are known from measurements, the impact direction is known through equation (1), while the impact location can be determined as follows:

From equation (2), uᵣ=uₐ×uₐ; and from equation (4) magnitude of vector r

mod(r)=Iₘ*mod(\dot{ω})/(m*mod(a_{cg})); (5)

Therefore, vector r is completely determined, including position of its tip. The location of application of vector F can be found as an intersection of the line defined by the force vector, going through the tip of vector r, and the body (head) surface.

In light of the above, and as shown in FIG. 14E, the system may perform a method of determining a location and direction of an impact force. In one or more embodiments, the method 500 may include receiving linear and angular acceleration vectors of an impact at a reference point on the free body. (502) The method may also include establishing the direction of the impact as the direction of a linear acceleration vector (504). The method may also include establishing the location of the impact (506). The step may include calculating an arm vector originating at the center of gravity of the head and extending to a perpendicular intersection with a line of force. The method may also include calculating an intersection of the line of force with a surface of the free body. In one or more embodiments, the method of may be based on the assumption that the line of force is may or may not extend through the center of gravity of the free body or, more simply, the method may avoid the assumption that the force does extend through the center of gravity.

Accurate and precise impact sensing may allow for meaningful assessments particularly when combined with large amounts of data collection and supporting clinical assessments. While the study of the brain remains a complex subject, concussion symptoms may be described as disruptions to the brain's ability to process information, which can stem from disruptions to the electrical fields in the brain or damage to brain matter. Functional losses associated with disruptions to the electrical field may be recoverable while functional losses associated with damage to brain matter may be recoverable depending on the severity of damage, the body's ability to restore damaged areas, and the body's ability to re-route information through undamaged parts of the brain. In any of the above cases, clinical assessments by training staff, clinician's, and other human personnel after impact events can help to identify the type of impacts and other factors that can cause concussions.

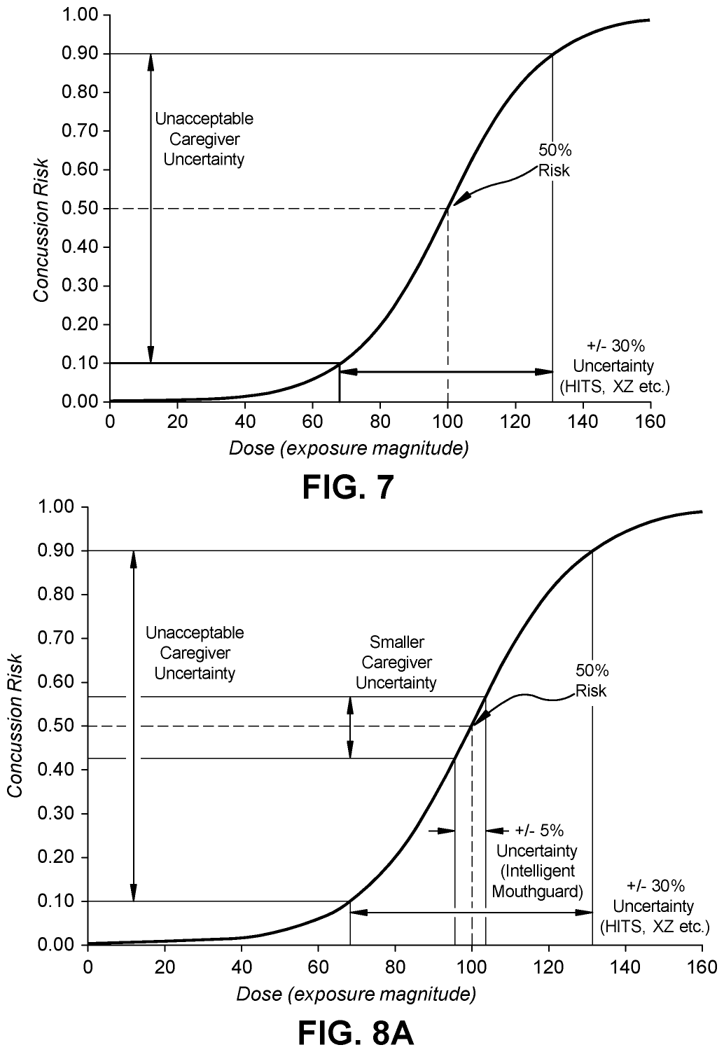

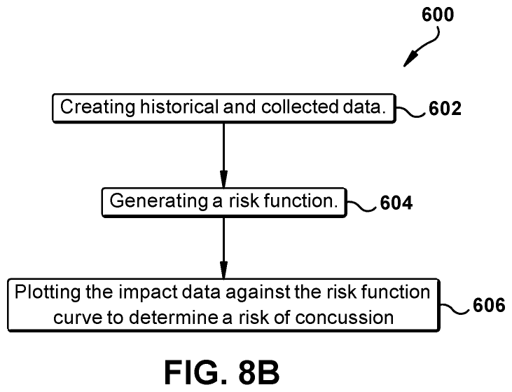

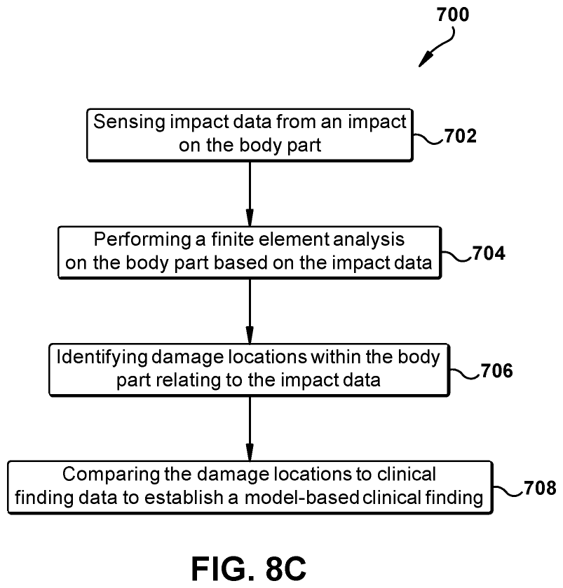

However, where impact measurements are insufficiently accurate or insufficiently precise, patterns are difficult to identify that allow for predictive assessments. That is, where the impact measurements are inaccurate or imprecise, impacts having similar sensed results may correspond to differing and/or opposite clinical assessments such as “concussed” and “not concussed.” As such, high degrees of uncertainty with respect to predictive assessments may exist based on previous data. For example, previous systems may have been focused merely on peak linear acceleration of an impact for example. However, depending on the location and direction of the impact (e.g., consider woodpeckers and big horn sheep), the impact may have a lesser or greater effect. An example of the type of risk certainty that may be present based on previous approaches is shown in FIG. 7. Rather, the present application focuses on more detailed impact results that take into account, among other things, translational and rotational acceleration, impact direction and location, and pre-conditioning effects relating to a lesser ability to resist head impacts due to prior exposure. FIG. 8A shows how the level of uncertainty may be reduced giving caregivers a better idea of the likelihood of injury and allowing for more appropriate responses to impacts. Methods described herein may allow for the use of historical and/or collected impact data to generate risk curves based on a variety of factors and to quickly assess a single hit or multiple hits to a user based on the risk curve. The risk curve may be a personal risk curve taking into account personal attributes, features, and a particular impact or series of impacts or a normative/population-based risk curve taking into account average attributes and features, but a personal impact or series of impacts. The historical and/or collected impact data may be from a broad range of users. Alternatively, or additionally, the historical and/or collected impact data may be from a single user including the user currently being monitored.

In one or more embodiments, the historical or collected data may include impact data from a large population of users or from a single user that includes impact direction and magnitude that may be broken down into orthogonal components such as X, Y, and Z. In addition, the historical or collected data may include linear acceleration, angular acceleration, linear velocity, and angular velocity. Where particular portions of the brain are found to be particularly relevant or susceptible to the effects of impacts, the particular forces or kinematics at that portion of the brain may be calculated by transferring the kinematics and/or forces and such data may be stored. In one or more embodiments, this may include the center of gravity of the head. The location of impact and the direction of the impact may also be stored. Still other factors that may be relevant to the effects of impacts may include age, sex, height, weight, race, head size, head weight, neck size, neck strength, neck girth or thickness, body mass index, skull thickness, and strength and/or fitness index, for example. Still other factors may be included that may have relevance to the effect of head impacts.

In addition to the above factors, cumulative impacts may also be collected and stored. In one or more embodiments, cumulative impacts may be processed with a fatigue-life calculation (e.g., a number of cycles at a given input energy), an energy model (e.g., a combination of linear velocity and angular velocity), an impulse-momentum model, a work-based model, restitution apart from energy, or an accumulated kinematics model. In one or more embodiments, a combination of these approaches may also be used. Still other models that may account for multiple impacts over time may be used. That is, while single impacts over particular thresholds that occur at particular locations, with particular directions, or that result in particular linear or angular accelerations may still be relevant for purposes of assessment, smaller impacts incurred over time may also be relevant. In one or more embodiments, periods of time may include same-day impacts, impacts occurring within a week, a month, a season, a year, or even a lifetime, for example. It is to be appreciated that particular windows of time may be selected and relevant windows of time may become more apparent when sufficient data is available to begin to understand the effects of cumulative impacts on clinical assessments.

In one particular embodiment, the cumulative effect of impacts may be a particular energy-based model that provides a scalar metric that captures a total effect of all head impacts received by an athlete over a chosen period of time. In particular, for example, the energy of an impact may be expressed as:

Eᵢ=(½mv²+½Iw²),
where:
m=mass
v=velocity
I−mass moment of Inertia; and
w=angular velocity.
   As may be appreciated, this energy equation takes into account both linear and angular velocities. To aggregate the energy from multiple impacts, the energy from a group of impacts may be added together. In one or more embodiments, each energy value from each impact may be adjusted using an aging factor to give older impacts lesser weight. In one embodiment, the cumulative effect scalar (S) may be calculated as follows:

S=Σ^{N}₁ n_p(kᵢ*Eᵢ)
where, N is the total number of impacts;
kᵢ is an aging factor to give older impacts lesser weight; and
Eᵢ=energy from impact number i.
n_p−a normalizing factor that can compare persons of different age, sex, weight, height, sport, helmet, race, genetics, etc.

The value kᵢ may range from 0 to 1, for example. However, where past impacts age poorly and, for example, have more effect as they age, the factor may be greater than 1. Still other values of kᵢ may be used.

It is to be appreciated that energy at a given point in time may be helpful as well as the power of an impact, which may be computed as the rate of change of the energy over time. While the instantaneous energy is detailed here, the power may be determined, accumulated, and stored as well. For example, since helmeted impacts (e.g., softer, longer contact time, more energy/power) may be different than bare head hits even with comparable accelerations, the effect of each of these may differ.

In one or more embodiments, the historical and collected data may include clinical assessments. For example, the assessment may include an assessment that is based on behavioral deficits and results in a diagnosis of concussed or not concussed. In the case of not concussed, there may still be a period of monitoring that is instructed based on the behavioral deficits and such may be part of the historical and collected data as well. Based on the clinical assessment, values of likelihood of concussion may be assigned such as 25%, 50%, 75%, or 100%, for example. In one or more embodiments, behavioral deficits themselves may be document and recorded or they may simply be part of the information that leads to the clinical assessment. In one or more embodiments, the behavioral deficits may include items such as balance, memory, attention, reaction time, and the like. In one or more other embodiments, and in addition to behavioral deficits, information relating to blood biomarkers, advanced imaging, advanced behavioral deficits, hydration, glucose levels, fatigue, heart rate, age, sex, race, height, weight, genetics, and other parameters may be taken into consideration and/or documented as relevant to the effect of an impact.

While all of the above factors may be recorded and stored for purposes of establishing a robust set of historical and collected data, in one or more embodiments, a method of assessing an impact may be based on spacial thresholds, temporal thresholds, and kinematics-based thresholds. In particular a parameter may be established that is based on 1) amplitude, frequency, and phase of translational and rotational accelerations and velocities and displacements, 2) shape and duration of the load pulse, and 3) the location and direction of the impact acting on the skull. That is, a point may be selected from any of the XYZ linear acceleration, angular acceleration, linear velocity, angular velocity in the time domain or frequency domain. For example, we may select (1) peak acceleration at the center of gravity, (2) kinetic energy at the time of peak acceleration at the center of gravity, and (3) this acceleration and kinetic energy transfer to a given direction and location on the skull.

In one or more embodiments, the historical and collected data may be used to create risk functions. For example, a risk function involving binary classification may be used where the binary part is OK or not likely OK. In one or more embodiments, the risk function may include a risk curve such as a logistic regression curve. For example, an S curve between binary data sets where 0=no injury and 1=injury may be generated using logistic regression. In one or embodiments, the curve may be a step function. Still other risk functions may include linear regression, receiver operating curve, decision trees, random forests, Bayesian networks, support vector machine, neural networks, or probit model.

As mentioned, in one or more embodiments, the risk function may be a risk curve. More particularly, in one or more embodiments, the curve may be a normalized (population-based) risk curves. In this embodiment, for example, user parameters may be used to classify the user into a particular population and the average parameters for that population may be used to develop risk curves for comparing individual impacts or a series of impacts. In one or more other embodiments, a personalized risk curve may be developed. In this embodiment, for example, individualized risk curves may be developed base on a user's particular attributes and individual impacts or a series of impacts may be compared to the individualized risk curves. In one or more embodiments, the risk curves for the individualized case may be based on population-based historical data or personal data of the user.

In one or more embodiments, as shown in FIG. 8B, a method 600 of assessing a user may be provided. The method may include creating historical and collected data by equipping a plurality of users with impact sensing mouthguards capable of sensing a variety of kinematics including linear acceleration, angular acceleration, linear velocity, angular velocity, displacement and the like. (602) The mouthguards may be configured for adequate coupling to users's upper teeth and may be equipped with some level of false positive protection and some level of co-registration so as to deliver accurate and precise kinematic readings. The users of the system may also be surveyed or required to enter other parameters into the system such as age, sex, weight, or any of the above-listed attributes. Impact readings may be collected over time and clinical assessments of injured players may be performed. Clinical assessment results may be entered into the system and associated with particular sets of impact and player/user attributes. Still further, each impact may be analyzed to determine other relevant parameters such as location and direction of impact, kinematics or forces at particular parts of the head, etc. and such calculated parameters may be stored in the database.

In one or more embodiments, the method may include assessing a user based on risk curves generated from the historical and collected data. It is to be appreciated that while the historical and collected data may be developed to a point where it is sufficient to begin using it for assessments, later impacts and assessments (including impacts being assessed with risk curves based on the historical and collected data) may continue to be used to populate and improve the historical and collected data.

Assessing a user based on risk curves may include generating risk curves. (604) For example, in one or more embodiments, a risk curve may be generated based on linear acceleration at a particular point in the head of a user and based on impacts occurring at a particular location. The values used to generate the risk curve may be values that relate to the impact being assessed. For example, all of the impacts involving an impact to the side of the head and exceeding a particular linear acceleration at the center of the brain may be plotted if the impact being assessed was to the side of the head and exceeded the selected threshold. The curve may include risk of concussion on a vertical axis and magnitude of linear acceleration on a horizontal axis. The plot may include a lot of data points showing low to zero likelihood of concussion near the lower linear acceleration values, an area of 25-75% likelihood of concussion as the acceleration increases and an area of 100% likelihood of concussion as the acceleration exceeds a higher value. The data may be fitted using equation fitting applications and curves similar to those shown in FIGS. 7 and 8 may be generated based on the data. Of particular note is that the more factors that play into the creation of the risk curve, the higher the likelihood that the risk is close to a true level of risk and the lower the uncertainty may be with the assessment. For example, the above-described curve may also be focused on a particular age group, a particular weight range, etc. Still further, multiple risk curves may be generated. For example, where the impact being assessed involves high levels of angular acceleration, risk curves based on angular acceleration may be generated as well. Finally, cumulative impacts may be included by focusing the risk curve on impact data where the clinical assessments have a energy scalar value exceeding a particular amount. It is to be appreciated that standard population risk curves may be generated based on the data and, in particular, where particular factors begin to be more relevant to concussion risk than others. However, standard risk curves may continue to change over time as more and more data is collected so while the parameters to generate the curve may be standard, the actual shape of the curve may continue to change.

Once the risk curve is established, the impact data from the present impact or series of impacts may be plotted against the curve to determine a risk of concussion, for example. (606) Still other approaches to creation of risk curves may be used based on the wide array of data in the historical and collected data database and based on the users being assessed.

In one or more embodiment, impact data may be used to predict brain damage and/or location of damage. For example, the accuracy/precision of the impact monitor data may allow for determinations of brain acceleration/force throughout the brain (i.e., at any location in the head). This may allow for a determination of what portion of the head experienced the highest accelerations and/or highest force and, thus, the location most likely to be damaged. Implementation methods may use data in a finite element model (FEM) to assess and/or determine brain damage. Using this approach, a prediction—substantially immediately post-impact—may identify a likely location of brain damage or injury. In one or more embodiments, this may involve the use of deformable body calculations and good material properties for the models.

In one method, a user-specific head FEM could be used or a normative head FEM could be used. Variances on the impact data may also be used to predict the most damaging impact types and the least damaging impact types. These types of models may inform us on how to design countermeasures, such as concussion-proof padding/helmets. Comparison of user-specific acceleration, algorithmically translated to head CG, vs. accelerometer data from a generic location shows that estimate of impact severity just by resultant acceleration magnitude may be insufficient. In one or more embodiments, a method may include using the head impact kinematic data (rigid skull movement) as a time dependent boundary condition in the brain injury model to identify risk of local tissue level injury. Then the time traces for X, Y, and Z linear and angular acceleration components may be used to adequately describe the skull kinematics. Knowledge of user-specific sensor positions and orientations with respect the athlete's head CG in a SAE J211 coordinate system, as well as algorithmic correction for non-linearities in sensor signals may be used. Spatial and temporal parameters of an impact may provide reasonable estimates of the skull kinematics for brain injury dynamic modeling.

The impact force vector magnitude, location, and direction change over time may be provided. Tracking the changing impact force vector may be advantageous for brain injury modeling or future experimentation. Accurate estimates of the impact location and direction, and of the impact force vector, from kinematic data are difficult to make throughout the full duration of impact and consequent recovery due to unknown neck restraining forces. However, for the initial stages of impact such estimates may be reasonably accurate in our laboratory calibration tests.

Analysis of free body force and moment balance at the time of peak acceleration (with negligible neck restraining force) shows that presumed impact force line of action does not necessarily go through head CG (see parameter ‘r’ in Error! Reference source not found. 17, uncertainty from 3 to 5 mm). However, such estimates rapidly lose meaning after the acceleration decreases down to zero from its peak value as the post-impact head motion moves.

In one or more embodiments, a finite element analysis may be performed to assess a user. For example, a method 700 of assessing an impact on a body part may include sensing impact data from an impact on the body part (702) and performing a finite element analysis on the body part based on the impact data (704). The method may also include identifying damage locations within the body part relating to the impact data (706) and comparing the damage locations to clinical finding data to establish a model-based clinical finding (708).

It is to be appreciated that many methods have been described herein that may have logical and practical application on a computer chip or other circuitry embedded in a mouthguard, other oral appliance, or another device capable of adequate coupling with the head of a user or other portion of a user. As such, while the methods have been described as such, many of the methods may be suitable as part of a sophisticated mouthguard or smart mouthguard, for example. That is, with very few exceptions, any of the methods described herein may be part of the circuitry of a mouthguard or other oral appliance. The exceptions may be methods involving other equipment such as MRI equipment, CT equipment, scanners, or other equipment not embeddable in an oral appliance. Other exceptions may include methods that simply are not programmable and require human performance or, as mentioned, performance of other equipment. Nonetheless, even when other equipment is used to perform a method, particular parts of pieces of the method may be part of the mouthguard.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a minicomputer, mainframe computer, personal computer (e.g., desktop or laptop), tablet computer, embedded computer, mobile device (e.g., personal digital assistant (PDA) or smart phone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as digital and analog general purpose I/O, a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, I2C, SPI, USB, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer, server, controller, or message sign, or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1-20. (canceled)

21. A method of assessing head impacts, comprising:
sensing impact data resulting from an impact to a user;
generating a risk function from a set of historical and collected data including other impacts and clinical assessments; and
plotting the impact data against the risk function to arrive at an assessment of the user.

22. The method of claim 21, further comprising wherein generating a risk function comprises basing the risk function, in part, on a measure of cumulative impacts over a selected period of time.

23. The method of claim 22, wherein the measure of cumulative impacts comprises an aging factor.

24. The method of claim 23, wherein a normalizing factor is used.

25. The method of claim 22, wherein the measure of cumulative impacts is based on an at least one of:
a) accumulation of the kinetics or kinematics of each impact;
b) impulse momentum and accumulation of momentum over time;
c) restitution apart from energy;
d) work energy; and
e) accumulated kinematics.

26. The method of claim 21, wherein the risk function is a normative risk curve.

27. The method of claim 21, wherein the risk function is a personalized risk curve.

28. The method of claim 21, wherein the risk function incorporates factors including one or more of the following:
Age;
Sex;
Weight;
Height;
Race;
Head size;
Head weight;
Neck size;
Neck strength;
Neck girth or thickness;
Body mass index;
Skull thickness; and
Strength and/or fitness index.

29. The method of claim 22, wherein the selected period of time includes at least one of:
a length of a game or event;
a day;
a week;
a month;
a season;
a year; or
a lifetime.

30-52. (canceled)

53. A method for calculation of impact location and direction on a rigid, free body, comprising:
receiving linear and angular acceleration vectors of an impact at a reference point on the free body;
establishing the direction of the impact as the direction of a linear acceleration vector;
establishing the location of the impact by:
calculating an arm vector originating at the center of gravity of the head and extending to a perpendicular intersection with a line of force; and
calculating an intersection of the line of force with a surface of the free body.

54. The method of claim 53, wherein the line of force is not assumed to extend through the center of gravity of the free body.

55. A method of assessing an impact on a body part, comprising:
sensing impact data from an impact on the body part;
performing a finite element analysis on the body part based on the impact data;
identifying damage locations within the body part relating to the impact data; and
comparing the damage locations to clinical finding data to establish a model-based clinical finding.
